(19) United States
(12) Patent Application Publication
(10) Pub. No.: US 2011/0319947 A1
(43) Pub. Date: Dec. 29, 2011

(54) Portable Low-Frequency Electrotherapy Apparatus

(75) Inventors: Youngsam Chun, Wonju-si (KR); Hanyoon Choi, Wonju-si (KR); Ki-jung Kim, Seoul (KR); Dongwoo Kim, Seoul (KR)

(73) Assignee: ELBIO CO., LTD., Wonju-si (KR)

(21) Appl. No.: 12/883,753

(22) Filed: Sep. 16, 2010

(30) Foreign Application Priority Data
Jun. 29, 2010 (KR) 10-2010-0061901

(51) Int. Cl. A61N 1/36

(52) U.S. Cl. 607/2

(57) ABSTRACT

The present invention relates to a portable low-frequency electrotherapy apparatus comprising a main body which comprises a universal serial bus (USB) port, a charging part to receive a charging voltage from the power pin of the USB port and a low-frequency signal generator electrically connected to a data pin of the USB port to supply a low-frequency signal through the data pin of the USB port; a low-frequency signal cable which comprises a low-frequency electrode connector at one end of the low-frequency signal cable electrically connected to the data pin of the USB port and an electrode pad at the other end to transmit a low-frequency signal to a human body; and a power cable which comprises a power supply connector at one end of the power cable electrically connected to the power pin of the USB port.

### CROSS-REFERENCE TO RELATED APPLICATION

This application claims priority from Korean Patent Application No. 10-2010-0061901, filed on Jun. 28, 2010 in the Korean Intellectual Property Office, the disclosure of which is incorporated herein by reference.

### BACKGROUND

1. Field

Apparatuses and methods consistent with the exemplary embodiments relate to a low-frequency electrotherapy apparatus, and more particularly, to a portable low-frequency electrotherapy apparatus which has a rechargeable battery therein and has an improved connector configuration not to perform an electrotherapy while recharging the battery.

2. Description of the Related Art

A low-frequency electrotherapy apparatus applies a low-frequency electric signal to a specific region of the human body for the purposes of compulsory exercise of the muscles, massage, pain relief, and losing weight. The low-frequency electrotherapy apparatus is widely used in medical facilities, and recently, household low-frequency electrotherapy apparatuses and portable low-frequency electrotherapy apparatuses are becoming more common.

FIG. 1 illustrates an example of a conventional portable low-frequency electrotherapy apparatus.

Referring to FIG. 1, the conventional portable low-frequency electrotherapy apparatus includes a main body 1 which generates a low-frequency signal, attachment pads 2 which are attached to a specific region of the human body, and a cable 3 which connects the main body 1 and the attachment pads 2.

For example, if a user operates the main body 1 after attaching the attachment pads 2 to the pain region, a low-frequency voltage or current from the main body 1 is transmitted to the pain region through the cable 3 and the attachment pads 2, and a strength of the signal is adjusted by a manipulation button (not shown) provided in the main body 1.

However, such a conventional portable low-frequency electrotherapy apparatus is designed to replace batteries in most cases. Accordingly, a power port and a cable are not needed any more, and the low-frequency electrotherapy apparatus can be smaller, lighter and more portable, thereby allowing manufacturers to provide high-end designs meeting customers' taste. Still, customers cannot but bear financial burden due to a frequent replacement of batteries.

The above problem may be solved if the electrotherapy apparatus employs a rechargeable secondary battery instead of normal batteries. However, in that case, the effect of using the normal batteries disappears, and it cannot be said that using the secondary batteries are far better than using the normal batteries.

More specifically, a charging port and a cable are needed for using the secondary battery. The increased number of parts leads to increase in costs, and, among others, the charging port ruins the design of a small electrotherapy apparatus. Further, this may cause upsizing of the electrotherapy apparatus which should be attached to underwear not to be seen by others.

Safety evaluation institutions have consistently raised an issue that a user's safety is not guaranteed if he/she conducts an electrotherapy and recharges the battery at the same time. In this respect, providing an additional power port was a burden to manufacturers.

Accordingly, manufacturers of conventional portable low-frequency electrotherapy apparatuses do not dare to employ the secondary battery instead of batteries.

### SUMMARY

Accordingly, one or more exemplary embodiments provide a portable electrotherapy apparatus which has a charging port, but prevents upsizing, concurrent conduct of recharging a battery and low-frequency electrotherapy and prevents the charging port from ruining the design.

Additional aspects and/or advantages of the present invention will be set forth in part in the description which follows and, in part, will be obvious from the description, or may be learned by practice of the present invention.

The foregoing and/or other aspects may be achieved by providing a portable low-frequency electrotherapy apparatus comprising a main body which comprises a universal serial bus (USB) port, a charging part electrically connected to a power pin of the USB port to receive a charging voltage from the power pin of the USB port and a low-frequency signal generator electrically connected to a data pin of the USB port to supply a low-frequency signal through the data pin of the USB port; a low-frequency signal cable which comprises a low-frequency electrode connector at the one end of the low-frequency signal cable to be connected to/disconnected from the USB port and electrically connected to the data pin of the USB port and an electrode pad at the other end transmit a low-frequency signal to a human body; and a power cable which comprises a power supply connector at one end of the power cable to be connected to/disconnected from the USB port and electrically connected to the power pin of the USB port, and a source connector at the other end to be electrically connected to an external power supply device.

According to an aspect of the invention, the low-frequency electrode connector is electrically insulated from the power pin of the USB port of the main body, and the power supply connector is electrically insulated from the data pin of the USB port of the main body.

According to another aspect of the invention, the source connector comprises a USB connector to be connected to a device such as a computer and to be charged thereby.

The foregoing and/or other aspects may be achieved by providing A portable low-frequency electrotherapy apparatus comprising a main body which comprises a port having two power pins and two data pins, a charging part electrically connected to the power pins of the port to receive a charging voltage from the power pins, and a low-frequency generator electrically connected to the data pins of the port to supply a low-frequency signal through the data pins of the port; a low-frequency signal cable which comprises a low-frequency electrode connector at one end of the low-frequency signal cable to be connected to/disconnected from the port and electrically connected to the data pins of the port, and attachment pads at the other end to transmit a low-frequency signal to a human body; and a power cable which comprises a power supply connector at one end of the power cable to be connected to/disconnected from the port and electrically connected to the power pins of the port and a source connector at the other end to be electrically connected to an external power supply device.

### BRIEF DESCRIPTION OF THE DRAWINGS

The above and/or other aspects will become apparent and more readily appreciated from the following description of the exemplary embodiments, taken in conjunction with the accompanying drawings, in which:

FIG. 1 illustrates elements of a conventional portable low-frequency electrotherapy apparatus;

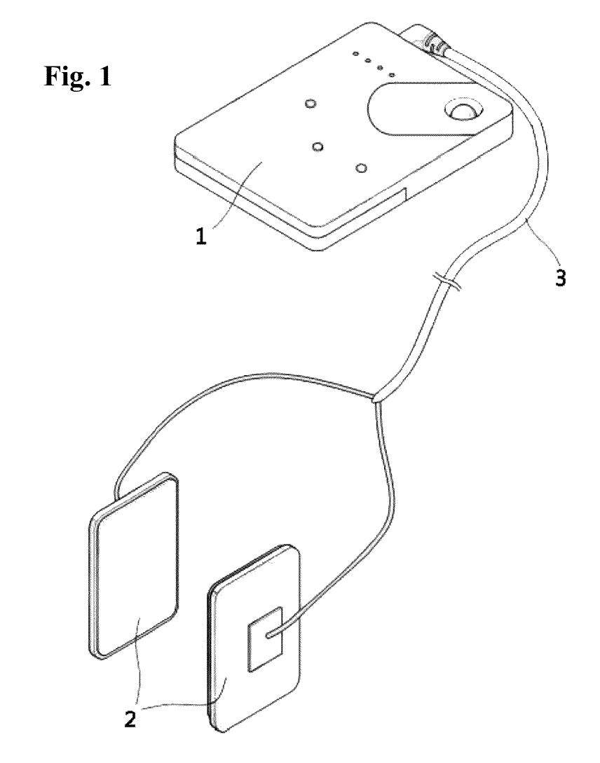

FIG. 2 illustrates elements of a low-frequency electrotherapy apparatus according to an exemplary embodiment of the present invention;

FIG. 3 is a rear view of a main body of the low-frequency electrotherapy apparatus according to the exemplary embodiment of the present invention;

FIG. 4 is a lateral view of the main body of the low-frequency electrotherapy apparatus according to the exemplary embodiment of the present invention; and

FIG. 5 is a block diagram of the main body of the low-frequency electrotherapy apparatus according to the exemplary embodiment of the present invention.

### DETAILED DESCRIPTION OF EXEMPLARY EMBODIMENTS

Below, exemplary embodiments will be described in detail with reference to accompanying drawings so as to be easily realized by a person having ordinary knowledge in the art. The exemplary embodiments may be embodied in various forms without being limited to the exemplary embodiments set forth herein. Descriptions of well-known parts are omitted for clarity, and like reference numerals refer to like elements throughout.

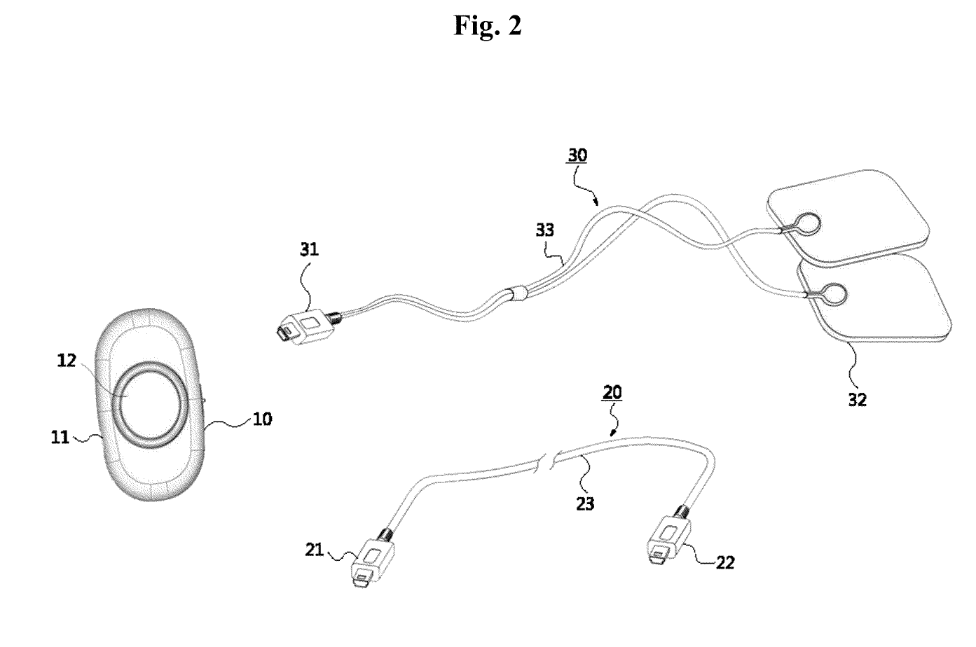

FIG. 2 illustrates elements of a portable low-frequency electrotherapy apparatus according to an exemplary embodiment of the present invention. Referring to FIG. 2, the portable low-frequency electrotherapy apparatus according to the present invention includes a main body 10 having a universal serial bus (USB) port 11 and a manipulation button 12; a power cable 20 which is selectively connected to the USB port 11 of the main body 10; and a low-frequency signal cable 30.

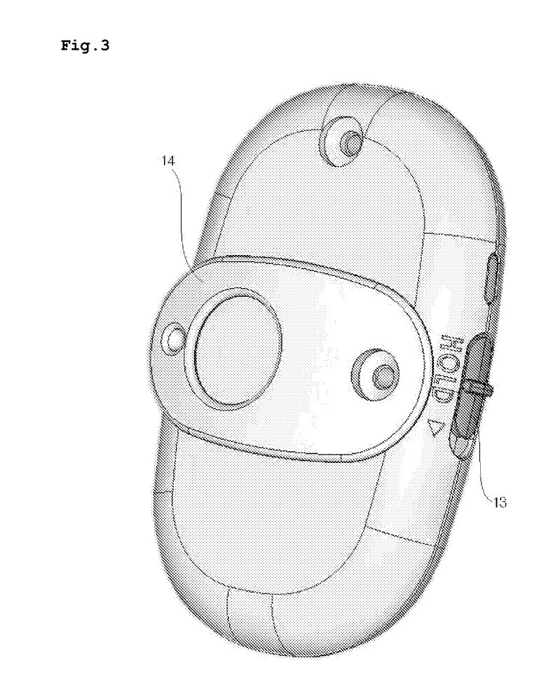

The manipulation button 12 which is provided in the main body 10 is designed as a round plate and has four buttons thereon. For example, if a left side of the round plate is pressed, the electrotherapy apparatus is turned on or turned off. If a right side of the round plate is pressed, a mode is selected. If an upper side of the round plate is pressed, the strength of a low-frequency signal becomes stronger, and upon pressing a lower side thereof, the strength of the low-frequency signal becomes weaker. As shown in FIG. 3, a hold button 13 is provided in an upper lateral side of the main body 10 to hold an operation of the low-frequency electrotherapy apparatus, and an attachment means 14 is provided in a rear side of the main body 10 to be attached to user's clothes.

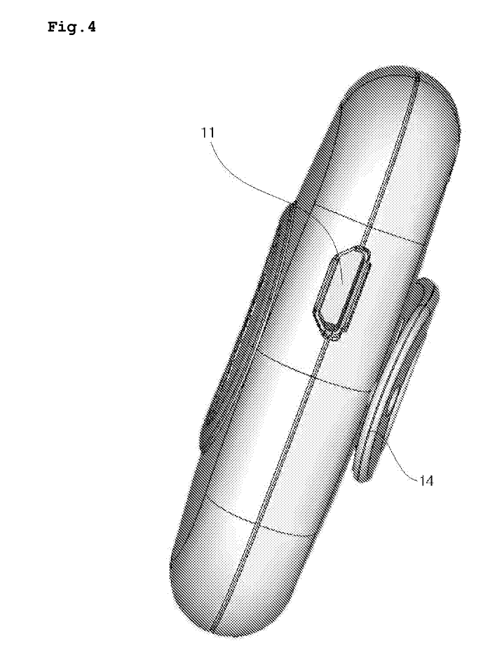

As shown in FIG. 4, only a single USB port 11 is provided in a lower lateral side of the main body 10. The USB port 11 includes four or five pins, and among others, at least two power pins and two data pins (not shown). The power pins are connected to a charging part 15 within the main body 10 and the data pins are connected to a low-frequency signal generator 16 within the main body 10.

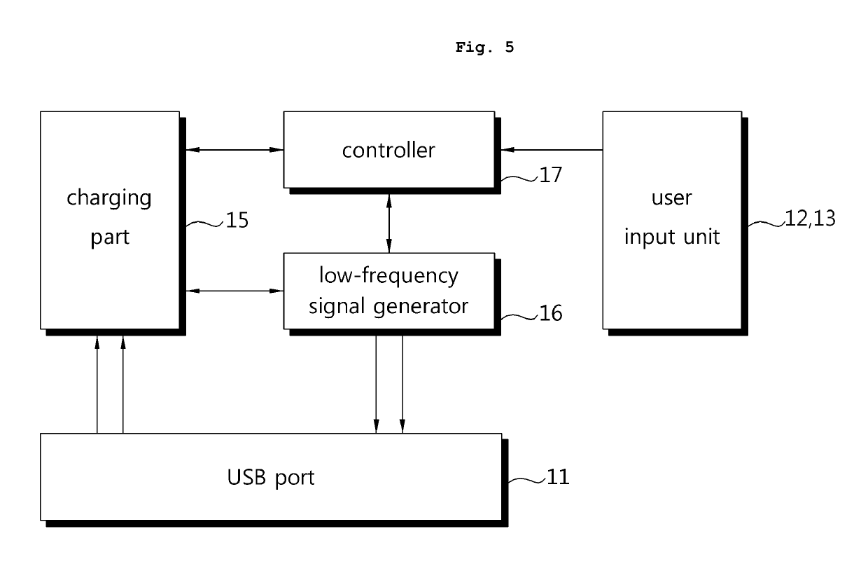

The internal configuration of the main body 10 will be described with reference to FIG. 5.

As shown in FIG. 5, the charging part 15 including a secondary battery and a charging circuit to charge the secondary battery; the low-frequency signal generator 16 which generates a low-frequency signal based on a voltage and a current provided by the secondary battery; a controller 17 which controls an overall operation of the portable low-frequency electrotherapy apparatus according to a user's input signal; user input units 12 and 13 which include the manipulation button 12; and the USB port 11 are provided within the main body 10.

The charging part 15 is electrically connected to the power pins of the USB port 11 provided in the main body 10, and charges a secondary battery (not shown) within the charging part 15 with the current supplied from the outside through the power pins.

The low-frequency signal generator 16 is electrically connected to the data pins of the USB port 11, and supplies the generated low-frequency signal to the outside through the data pins.

The controller 17 controls the operation of the portable low-frequency electrotherapy apparatus according to a user's command input through the user input means within the main body 10. For example, if a user presses a switch to adjust a strength of a low-frequency signal, the controller 17 controls the low-frequency signal generator to generate a low-frequency signal in a strength corresponding to the user's input or controls power to be supplied to, or cuts down power supplied to, the secondary battery when a power on/off signal is input.

Returning to FIG. 2, remaining two cables 20 and 30 other than the main body 10 will be described.

The power cable 20 has one end which is formed as a USB connector 21 to be connected to the USB port 11, and the other end which is formed as a USB connector 22 or a plug to be connected to an external power source. In this case, the data pins of the USB port 11 are used as a terminal outputting a low-frequency signal generated by the internal low-frequency signal generator 16. Thus, the USB connector 21 which is connected to the USB port 11 of the main body 10 is preferably insulated from the data pins. If the USB connector 22 is used as a connector to be connected to an external power source, a personal computer (PC) may be such an external power source. If a plug is used to be connected to the external power source, a charger such as an adapter for charging a mobile phone may be connected to a 220V commercial power source.

The low-frequency signal cable 30 has one end which is formed as a USB connector 31 to be connected to the USB port 11, and the other end which is formed as attachment pads 32 to be attached to a user's body. The USB connector 31 and the attachment pads 32 are connected by a cable 33 which transmits a low-frequency signal. The power pins of the USB port 11 of the main body 10 are used as a terminal to receive a charging voltage or current from the external power source, and the USB connector 31 which is connected to the USB port 11 of the main body 10 is preferably insulated from the power pins.

A user should first charge the secondary battery of the main body 10 to use the low-frequency electrotherapy apparatus. Accordingly, a user connects the USB connector 21 of the power cable 20 to the USB port 11, and connects the USB connector 22 or the plug at the other end of the power cable 20 to the power source to start charging the secondary battery.

If the charging operation is completed, a user disconnects the power cable 20 from the USB port 11 and connects the low-frequency signal cable 30 to the USB port 11 of the main body 10.

Then, a user attaches the low-frequency electrotherapy apparatus to his/her underwear by the attachment means 14 and attaches the attachment pads 32 to the area in which pain is anticipated. A user then presses the manipulation button 12 and starts the operation of the low-frequency electrotherapy apparatus and properly adjusts the strength of the low-frequency signal whenever he/she wants to.

For example, a female user may use the low-frequency electrotherapy apparatus to relieve period pains. If the period pains are too severe, a female user may press the manipulation button 12 and applies the low-frequency signal to the pain area to relieve such pains. A female user may adjust the strength of the signal by pressing the manipulation button 12.

Considering sensitivity of women, the design of the low-frequency electrotherapy apparatus is a major factor directly connected to sales, and the low-frequency electrotherapy apparatus should be small for a female user not to feel ashamed.

If a user intends to use the low-frequency electrotherapy apparatus at home after the battery is all discharged, he/she might charge the battery and conduct the low-frequency electrotherapy at the same time, which may cause electric shock. In that respect, it is necessary to force a separation of the charging the secondary battery and conduct of the low-frequency electrotherapy for the purposes of safety.

According to the present invention, the main body 10 has only one USB port 11 formed therein to make the electrotherapy apparatus downsized while ensuring the charging function by the secondary battery. Accordingly, when a user attaches the electrotherapy apparatus according to the present invention to his/her underwear, the electrotherapy apparatus is not seen from the outside and maintains a superior design. Further, the electrotherapy apparatus according to the present invention has one USB port 11 jointly used for the purposes of charging the battery and conducting the electrotherapy, thereby preventing charging and electrotherapy from being conducted at the same time. Thus, any accident due to a user's negligence may be prevented.

The electrotherapy apparatus according to the exemplary embodiment of the present invention has been described and the skilled in the art may fully understand that other exemplary embodiments are available to the extent not exceeding the spirit of the present invention.

For example, the connectors 21 and 31 which are connected to the USB port 11 may not have the power pins or data pins formed therein depending on the purpose of the cables 20 and 30. Otherwise, the connectors 21 and 31 may have four or five pins, and the connection of conductive lines connected to a particular pin within the cables 23 and 33 may be omitted.

Further, other types of four pin connectors and ports may be used instead of the USB connector and the USB port. In this case, the electrotherapy apparatus may not receive power from a device such as a computer through the USB.

As described above, a low-frequency electrotherapy apparatus according to the present invention has a physical configuration which prevents a user from conducting charging a battery and an electrotherapy at the same time and prevents any accident which may occur when the charging and the electrotherapy are conducted at the same time.

Also, a low-frequency electrotherapy apparatus according to the present invention does not need any additional charging port since one port is jointly used for a low-frequency electrotherapy and charging the battery. Accordingly, the low-frequency electrotherapy apparatus may be small in size and flexible in design, thereby increasing user's satisfaction for the design and improving product competitiveness.

Although a few exemplary embodiments have been shown and described, it will be appreciated by those skilled in the art that changes may be made in these exemplary embodiments without departing from the principles and spirit of the invention, the scope of which is defined in the appended claims and their equivalents.

What is claimed is:

1. A portable low-frequency electrotherapy apparatus comprising:
a main body which comprises a universal serial bus (USB) port, a charging part electrically connected to a power pin of the USB port to receive a charging voltage from the power pin of the USB port and a low-frequency signal generator electrically connected to a data pin of the USB port to supply a low-frequency signal through the data pin of the USB port;
a low-frequency signal cable which comprises a low-frequency electrode connector at one end of the low-frequency signal cable to be connected to/disconnected from the USB port and electrically connected to the data pin of the USB port and an electrode pad at the other end to transmit a low-frequency signal to a human body; and
a power cable which comprises a power supply connector at one end of the power cable to be connected to/disconnected from the USB port and electrically connected to the power pin of the USB port, and a source connector at the other end to be electrically connected to an external power supply device.

2. The portable low-frequency electrotherapy apparatus according to claim 1, wherein the low-frequency electrode connector is electrically insulated from the power pin of the USB port of the main body, and the power supply connector is electrically insulated from the data pin of the USB port of the main body.

3. The portable low-frequency electrotherapy apparatus according to claim 1, wherein the source connector comprises a USB connector.

4. The portable low-frequency electrotherapy apparatus according to claim 2, wherein the source connector comprises a USB connector.

5. A portable low-frequency electrotherapy apparatus comprising:
a main body which comprises a port having two power pins and two data pins, a charging part electrically connected to the power pins of the port to receive a charging voltage from the power pins, and a low-frequency generator electrically connected to the data pins of the port to supply a low-frequency signal through the data pins of the port;
a low-frequency signal cable which comprises a low-frequency electrode connector at one end of the low-frequency signal cable to be connected to/disconnected from the port and electrically connected to the data pins of the port, and attachment pads at the other end to transmit a low-frequency signal to a human body; and
a power cable which comprises a power supply connector at one end of the power cable to be connected to/disconnected from the port and electrically connected to the power pins of the port and a source connector at the other end to be electrically connected to an external power supply device.
